(19) United States
(12) Patent Application Publication
(10) Pub. No.: US 2003/0219456 A1
(43) Pub. Date: Nov. 27, 2003

(54) Method of utilization of zygosaccharomyces rouxii

(76) Inventor: Taing Ok, Jagoshima (JP)

Correspondence Address: MALIN HALEY AND DIMAGGIO, PA, 1936 S ANDREWS AVENUE, FORT LAUDERDALE, FL, 33316, US

(21) Appl. No.: 10/153,209

(22) Filed: May 21, 2002

(51) Int. Cl. A61K 35/72; C12N 1/18

(52) U.S. Cl. 424/195.16; 435/254.2

(57) ABSTRACT

The present invention provides commercial utilization of a novel yeast strain Zygosaccharomyces rouxii and its fermented metabolites as probiotic, as an antioxidant and as an antimicrobial agent in foods and cosmetics. The fermented broth and metabolite substance (s) produced by this invention have a wide spectrum antibacterial activity, strong antioxidant activity, cytochalasin-like activity that inhibits cell cleavage, and is expected to be effective in the treatment of allergy, atopic dermatitis, psoriasis and various skin diseases, second degree burns, high blood pressure, diabetes, cancer, AIDS and as an anti-aging agent. Moreover, this invention is expected to lead to industrial utilization of Z.rouxii for manufacturing succinic acid and malic acid from a yeast.

### BACKGROUND OF THE INVENTION

1. Field of the Invention

This invention relates generally to the composition and utilization of yeast strain Zygosaccharomyces rouxii, hereinafter (Z.R. or Z. Rouxii) and its fermented metabolites in the field of human health and, specifically, as a composition for and use as an antioxidant and, independently, as an antimicrobial agent in foods and cosmetics.

2. Description of the Related Art

Yeast Z.rouxii is primarily regarded as a contaminant in high-sugar foods such as fruit and jam. On the other hand, it is a natural inhabitant in honey, wine and Japanese soy paste (miso), traditional foods since ancient times that are recognized for good health and long life.

Out of 500 plus species of yeast identified so far, only Saccharomyces cerevisiae has been employed commercially. There is little industry that utilizes Z.rouxii. A patent related to Z.rouxii is concerned only with the aroma of soy sauce (U.S. Pat. No. 5,210,034, issued May 11, 1993).

There has been a plethora of new antibiotic drugs as more and more drug-resistant strains have been appearing. Natural products, like the substances produced in this invention, that help the patients heal by themselves are desired.

Hundreds of synthetic antioxidant compounds have been used as additives in foods and cosmetics, most of which are not suitable for consumption as these components may produce undesirable side effects. Natural compounds of plant origin seem to be safe but cultivating plants requires a lot of time and commonly a considerable investment. By contrast, cultivation of microorganisms, particularly yeasts, is simple and relatively inexpensive, easy to control and, most of all, fast.

Organic acids such as malic acid, succinic acid and fumaric acid are not only important as industrial raw materials but also as major ingredients in many medicines, food supplements and sport or health drinks. These acids are widely accepted as anti-microbial agents and antioxidants. Although these organic acids are widely distributed in nature, commercial production has been dependent largely on synthetic chemical methods. The safety of employing these synthetic organic acids in food, cosmetic and medicinal products is largely in question.

The following references are cited. Taing Ok & F. Hashinaga: Detection and production of extra-cellular collagenolytic enzyme from Zygosaccharomyces rouxii, J. Gen. Appl. Microbiol., 42, 517-523, 1996.

Taing Ok & F. Hashinaga: Isolation and identification of sugar-tolerant yeasts from high-sugar fermented vegetable extracts, J. Gen. Appl. Microbiol., 43, 39-47, 1997.

### SUMMARY OF THE INVENTION

It is an object of this invention to avoid safety problems by providing natural fermented products from a safe strain of Z.R. yeast for use as an antioxidant in foods and cosmetics.

A composition and method of providing safe antioxidants and antimicrobial agents in human foods and cosmetics by the utilization of Z.rouxii yeast strain, composition and method, through steps of exploiting the Z.R.'s distinct properties and metabolites of Z. rouxii yeast.

Z.rouxii is a robust osmo-tolerant yeast. It not only withstands high concentration of salt or sugar, some strains even show their optimum growth at higher concentrations. In one experiment, Z.rouxii V19 still grows in the medium containing 80% (w/v) of glucose. W/V denotes throughout, weight per volume in grams per millimeter.

Z.rouxii is a rich vitamin source and produces an abundant amount of B group vitamins.

The Z.rouxii mitochondria contains a coenzyme Q6, a strong antioxidant agent.

Some strains show the activity of hydrolyzing gelatin or native collagen, which provides the possible utilization of Z.rouxii for meat tenderization.

By optimizing the cultural conditions, some strains of Z.rouxii produce large amounts of metabolite succinic acid, malic acid and a small quantity of fumaric acid.

Succinic acid is a major acid in amber, traditionally accepted as a bio-stimulant that has a positive effect on all human organs, stimulates the nervous system, regeneration process, and functioning of the heart and kidneys. Succinate-coenzyme Q complex is one of the essential components of mitochondria in cells.

Malic acid is a natural constituent of many fresh and preserved fruits and vegetables, and is recognized to improve energy production in primary fibromyalgia (FM). Because of its obvious improvement of energy depletion during exercise, malic acid is beneficial to healthy individuals, like athletes, interested in maximizing their energy production, as well as those with FM. Current principal uses of malic acid includes food additives, feed additives, especially as an acidifier during pig weaning, pet foods, cosmetics, pharmaceuticals, and industrial uses such as metal cleaning, metal plating, and in textile industries.

Fumaric acid is an acidulant, antidermatitic, antihepatocarcinogenic, antioxidant, antipsoriac, and anti-tumor. It is an important starter for manufacturing of pharmaceutics, plasticizers, and industrial resin. It is also being used in the food industry as a flavoring agent and acidulant and also as animal feed. A small portion of fumarate esters, such as monoethyl fumarate, which is a reaction product of fumaric acid and ethyl alcohol, has been observed for its therapeutic ability for psoriasis, a chronic relapsing cutaneous disorder characterized by inflammation and increased epidermal proliferation with a prevalence of 2-3% in the general population. There are also reports of successful therapy with fumaric esters on disseminated granuloma annulare.

It is an object of this invention to avoid safety problems by providing natural fermented products from a safe strain of Z.R. yeast for use as an antioxidant in foods and cosmetics.

It is another object of the invention to provide natural fermented products, metabolites, from a safe strain of yeast (Z.R.) for use as a food preservative, antimicrobial agent, food supplment, commercial source of natural sucinnic acid, malic acid and fumaric acid.

But yet still another object of this invention is to provide a composition o f the yeast of Z.R. and the method of its use for sport and health drinks, food additives, anti-bacterial substances, anti-aging agents, and medicinal therapy agents.

### BRIEF DESCRIPTION OF THE DRAWINGS

FIG. 1 is a photograph of a meat preservation test, showing the action in Example 2.

VEL I, VEL II, VEL III: Vegetable enzyme liquid media, produced by this invention, varying only in their proportion of fruit used and thus in their protease activity.

Pa I, Pa II: Synthetic media having the same composition of sugar and acidity, with added commercial papain to get the same activity as the corresponding VEL products

Control: The same synthetic medium without added papain

FIG. 2: Phase-contrast microscope photographs of Microcoecus luteus (IFO 12708), 18 hours (18-h) incubation in nutrient broth medium of pH 5.5 at 30C (600 magnification), showing the action in Example 5.

(A) without added Sample (B) with 1 mg/ml Sample

FIG. 3: SEM photograph of Salmonella enteritidis (IFO 3313), 18-h incubation in nutrient broth medium of pH 5.5 at 30C with 2 mg/ml of Sample (10,000 magnification), showing the action of Example 5.

FIG. 4 SEM photograph of Eseherichia coil (IFO 3301), 18-h incubation in nutrient broth medium of pH 5.5 at 30° C. with 2 mg/mi of Sample (10,000 magnification) showing the action in Example 5.

FIG. 5 is a graph showing the growth of Micrococcus Luteus in accordance with Example 6.

FIG. 6: Light microscope photographs of Pseudomonas aeruginosa (IFO 12689), 18-h incubation in nutrient broth of pH 7.2 at 30C (600 magnification)

(A) Wild type cells (untreated)

(B) Mutant cells (treated with Sample for one month and restored)

FIG. 6a and FIG. 6b are photographs of the action as shown in Example 7.

FIG. 7: Photograph of colonies of Micrococcus Juteus (IFO 12708); many reddish yellow colonies observed with some wild type bright yellow colonies, showing the action from Example 8.

### PREFERRED EMBODIMENT OF THE INVENTION

In the preferred embodiment of this invention, the composition and method of use of Zygosaccharomyces rouxii is described specifically for its antioxidant properties. The following is an example of a Z.R. composition and utilization for its antioxidant properties in foods and cosmetics:

Zygosaccharomyces rouxii was first cultured. Strain Z.rouxii Cy7 was cultured in YMM medium (yeast extract, 2%; malt extract, 2%; polypeptone, 2%; glucose, 25%; KH₂PO₄, 4%; MgSO₄.7H₂O, 4%; all in w/v. The initial pH was adjusted to 4.0). The medium (150 ml) was dispensed in each of 500-ml Erlenmeyer flasks, fitted with silicon plugs and the flasks were autoclaved at 121° C. for 20 min. To each flask was inoculated with 3 ml of overnight grown suspension of inoculum (which was incubated at 30° C. in YM medium consisting of yeast extract, 0.3%; malt extract, 0.3%; polypeptone, 0.5%; glucose 1%; pH unadjusted). Inoculated flasks were put on a reciprocal shaker (80 strokes per min) inside an incubator of 30° C. and were incubated for 4 days.

The cultured broth was then extracted. The broth was harvested by centrifuging the fermented mass at 7000 rpm for 15 min in a refrigerated centrifuge. After cells were discarded, the broth was collected as a ZR drink to get the sample for further investigation. The pH of the ZR drink was first adjusted to 2.0 with 10 N HCl (N is the short form for Normality) and then it was extracted three times with equal volumes of ethyl acetate in a separating funnel. The combined organic layer was left overnight on Na₂SO₄ to remove traces of water and was evaporated to dryness under vacuum at 30° C. The dried mass was granular and was pale yellow. One litre of medium yielded 1.3 gm of dried product, which can be directly used as an antioxidant.

The product was then partially purified. The granular mass was re-dissolved in a few ml of ethyl acetate in a glass crucible and left overnight. The organic acids, mainly succinic acid, were crystallized out. The mother liquor, or magma was evaporated to dryness under vacuum and the dried mass was designated hereafter bio-polyphenols (BPP). The BPP was dissolved in ethyl alcohol to get concentrations of 100 mg/ml to 400 mg/ml. Standard compounds of BHA, BHT and vitamin E, which are recognized as strong antioxidants, and the ZR drink (as described in Example 4 below) were also employed for comparison. Concentration of standard compounds in ethyl alcohol was 100 mg/ml each.

BPP (684 mg), after being dissolved in 1 ml ethyl alcohol, was fractionated on silica gel column (Wako silica gel 300; column diameter, 1.2 cm; column height, 43 cm). The column was eluted with 500 ml (250 ml×2) each of acetone-hexane mixture (20:80, 40:60, 60:40, 80:20, and 100:0) and finally with 300 ml of methyl alcohol. Each fraction was evaporated to dryness and dissolved in ethyl alcohol to get a concentration of 0.1 g/ml.

Free radical scavenging activity was then evaluated. Standard solutions (0.1 g/ml in ethyl alcohol) of BHA, BHT and vitamin E were freshly prepared. DPPH scavenging activity was determined as described below. The following observations and results were then recorded:

| | Test | Blank | Control |
|---|---|---|---|
| Item | (ml) | (ml) | (ml) |
| BPP column fractions/Standards | 0.05 | 0.05 | 0.05 |
| Ethyl alcohol | 2.05 | 3.05 | 2.05 |
| 0.1 M acetate buffer, pH 5.5 | 1.0 | 1.0 | 1.0 |
| 0.5 mM DPPH solution in ethanol | 1.0 | 1.0 | 1.0 |

Optical density OD was measured at 517 nm at 0 min and 30 min after mixing. Percent free radical scavenging activity was calculated as follows:

Percent activity=100×{Ac1−(At2−Ab1)}/Ac2,

Where Ac1=OD of control at 0 min

At2=OD of test at 30 min

Ab1=OD of blank at 0 min

Ac2=OD of control at 30 min

The results are tabulated below:

| Test item | Percent activity |
|---|---|
| Sample, 100 mg/ml | 27.2 |
| Sample, 200 mg/ml | 52.7 |
| Sample, 300 mg/ml | 73.4 |
| Sample, 400 mg/ml | 88.4 |
| ZR | 61.8 |
| BHA | 83.0 |
| BHT | 83.4 |
| Vitamin E | 83.0 |

The above results prove that the product ZR drink and the substances extracted from ZR have high activity of scavenging free radicals and Z.R. and substances abstracted from Z.R. can be used as natural antioxidants for foods and cosmetics.

Peroxide value (POV) activity was determined by a slightly modified Rodan's method (Terasawa, N., Yamazaki, N., and Fukui Y: Antioxidant activity of water extracts of herbs, Nippon Shokuhin Kagaku Kogaku Kaishi, 48, 2, 99-104, 2001.)

| | Test | Control |
|---|---|---|
| Item | (ml) | (ml) |
| Substrate (1.3% linoleic acid in ethanol) | 1 | 1 |
| 0.2 M phosphate buffer, pH 7.0 | 1 | 1 |
| Test sample/Standards | 0.05 | 0 |
| Ethyl alcohol | 0 | 0.05 |
| Water | 0.1 | 0.1 |

After mixing in a screw-capped test tube, the reaction mass was incubated inside a dark incubator at 50° C. for 3 days. The assay of liberated peroxide compounds was carried out as follows.

| Item | Volume (ml) |
|---|---|
| Incubated reaction mass | 0.1 |
| 0.02 M FeCl₂ solution in 3.5% HCl | 0.1 |
| 30% ammonium thiocyanate | 0.1 |
| 75% ethyl alcohol | 3.7 |

After 3 minutes, optical density was measured at 500 nm. Percent POV activity was calculated as follows:

Percent activity=100×(OD of sample or standard/OD of control)

The results are tabulated below:

| Test item | Percent activity |
|---|---|
| 20:80 Fraction I & II combined | 54.2 |
| 40:60 Fraction I | 51.8 |
| 40:60 Fraction II | 41.2 |
| 60:40 Fraction I | 55.7 |
| 60:40 Fraction II | 57.6 |
| 80:20 Fraction I & II combined | 48.0 |
| 100:0 Fraction (insufficient weight) | Not tested |
| Methanol Fraction | 40.7 |
| BHA | 8.9 |
| BHT | 13.5 |
| Vitamin E | 0 |

The above results show that BPP can be further purified to get fractions with free radical scavenging activity much higher than that of standard compounds at very low concentrations and can be used as a natural antioxidant for foods and cosmetics.

In alternate embodiments, the composition of Zygosaccharomyces rouxii and its fermented metabolites and the method of its use show that the yeast can be used in the field of antimicrobial agents, food preservation and food supplements. The following specific examples are presented to afford a better understanding of alternate uses. It is understood that these examples are intended to better illustrate the invention and are not intended to limit the invention in any way.

### EXAMPLE 1

Vegetables, fruits, and herbs were washed and preserved with equal weight of sugar for one month and were pressed to get syrup. After inoculating with old stock starter or cultured inoculum of Z.rouxii, the syrup was fermented under controlled temperature for about two months with daily handling or aeration and occasional addition of more sugar. The fermented syrup was matured for about one month. The final pH of the product was about 4 with total soluble solid content of 55 to 60% and titratable acid content of 40 to 50 milligram equivalent per liter. Depending on the vegetables and fruits used, the flavor ranged from vegetable juice to fruit juice. With ripened product, it had a mellow honey flavor. The total count of yeast cells was 2-5×10⁷, of which viable count was 1-4×10⁶. The said product can be consumed straight or diluted with ice, alcohol, fruit juice, milk or water before drinking and can be stored for a few months at room temperature and for years at refrigerated temperatures. Alternatively, the said product can be distributed as food supplement in capsules or as concentrate.

### EXAMPLE 2

Three products of Example 1, called vegetable enzyme liquid (VEL), only varying in proportion of fruit used and thus in their protease activity, were tested for their preservative ability of meat. Three pieces of meat slices (2 cm×2 cm×1 mm) were placed in 20 ml each of the product of Example 1 in Petri dish. Synthetic medium containing the same sugar and organic acid composition with the same pH as VEL medium was used as control. Since the said products had a moderately high protease activity, another medium of the same composition as control medium but with added commercial papain protease was also used. The amount of added papain was adjusted so that the protease activity in the medium would be the same as in the said products medium. Triplicates were made for each medium. The Petri dishes were kept at 26° C. for up to one week during which 0.1 ml of drip from each dish was taken occasionally to determine the dissolved amino acids by Rosin's method. The results of drip loss after one week are described below.

| | Final weight of | Final drip | |
|---|---|---|---|
| Medium | 3 pieces of meat (gm) | (ml) | Amino acid loss in the drip |
| Control | 3.4 | 15.0 | 87.0 |
| VEL | 5.1 | 12.2 | 81.7 |
| Papain | 14 | 14.0 | 99.4 |

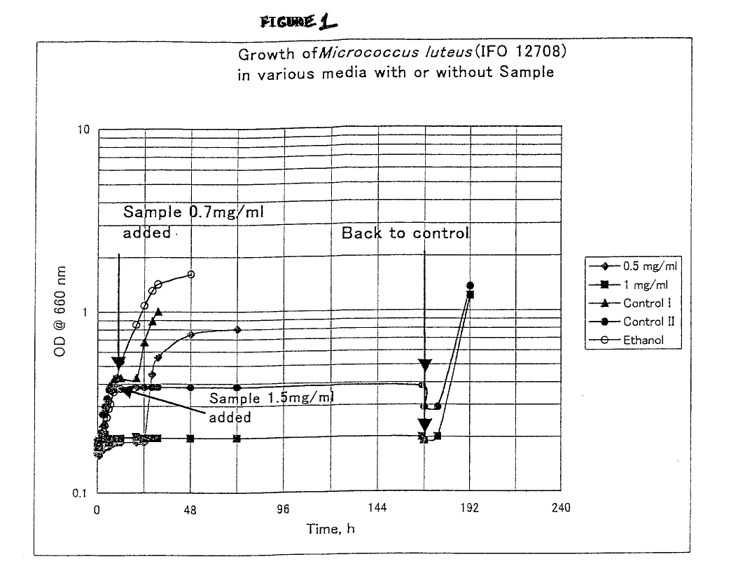

The observatory result of the appearance of meat slices is shown in FIG. 1 and described below. All meat slices did not putrifect.

Control: The meat slices shrank slightly. Meat color was pale yellow to light brown. The original color had faded out. The drip was cloudy and there were some oil droplets on the surface. Growth of fungus was observed on the surface of most of the meat slices.

VEL: The meat slices did not shrink; instead they became tendered and swelled a bit. Meat color was pale red to red; the original color of meat was mostly maintained. The drip was cloudy but no traces of oily droplets. No fungus was observed.

Papain: The meat slices shrank considerably. Meat color was light brown to dark. The drip was rather clear to partially cloudy, but there were oily droplets in the drip and growth of fungus was observed on the surface of some meat slices.

### EXAMPLE 3

Semi-aerobic stationary fermentation was carried out for 7 to 30 days at 20° C. to 30° C. in YPG medium (yeast extract, 0.5%; polypeptone, 1.0%; glucose 10 to 50%; all in w/v) of initial pH 4 to 8, with or without inclusion of mineral supplements and organic precursors. One ml of fermented broth was taken out periodically and ion-exchanged with amberlite IR-120 [H⁺] and amberlite IR-45 [OH⁻] resins in two connected columns successively. The latter column was eluted with ammonia solution. The ammonia solution was evaporated under vacuum to get dried substance, which was purified in ODS column. Purified substance was subject to HPLC and also to GC-MS, after it was methylated or TMS esterified. Malic acid, succinic acid and trace amount of fumaric acid were identified. Glucose concentration of 30%, initial pH of 5, incubation temperature of 25° C. and incubation time of 15 to 18 days were optimum conditions. Inclusion of 0.1% KH₂PO4 and 0.3% to 0.5% of precursors glutamic acid and malic acid enhanced the yield of malic acid considerably, and that of succinic acid slightly. Maximum malic acid concentration of 37.1 g/L was achieved with 0.5%-added malic acid to YPG medium with 30% glucose.

TABLE 1
Yield of malic and succinic acids based on sugar consumed

| % Glucose | Added | Acid produced (g/L) | | % Yield of acid | |
|---|---|---|---|---|---|
| in medium | supplement | Malic | Succinic | Malic | Succinic |
| 10 | 0.3% malic | 21.5 | 7.7 | 22.6 | 8.1 |
| 10 | 0.3% succinic | 10.1 | 6.1 | 10.6 | 6.4 |
| 10 | 0.3% glutamic | 17.8 | 5.8 | 18.2 | 5.9 |
| 30 | 0.5% malic | 37.1 | 6.8 | 21.1 | 3.9 |
| 30 | 0.5% succinic | 13.3 | 7.4 | 4.1 | 2.3 |
| 30 | 0.5% glutamic | 74.9 | 5.5 | 32.8 | 2.8 |

### EXAMPLE 4

Z.rouxii Cy7 was shake-cultured in a typical medium, pH adjusted, containing 20 to 30% glucose with mineral supplements, for 3 days at 30° C. and the fermented mass was centrifuged to discard the cells. The broth, which can be consumed as sport drink or food supplement, and designated as ZR drink, has the following properties:

| | |
|---|---|
| Appearance: | Crystal clear amber yellow |
| pH: | 2.9 to 3.5 |
| Specific gravity: | 1.01 to 1.04 |
| Total soluble solid: | 9 to 18 |
| Alcohol content: | 1 to 7% |
| Organic acids: | 60 to 70 mg/L |
| Bio-polyphenols: | 600 to 700 mg/L |

### EXAMPLE 5

The product ZR drink in Example 4 was extracted twice with an organic solvent, for example ethyl acetate, and the organic layer was evaporated completely to dryness under vacuum to get bioactive substances. One liter of the product drink yielded 1 to 1.5 g of bioactive substances (designated as Sample hereinafter) that was used in antibacterial assays. The Sample was added into a typical bacterial medium, concentration ranging from 0 mg/ml (control) to 4 mg/ml. Overnight grown suspension of the target pathogenic bacteria was inoculated into the said prepared medium and incubated at 30° C. for 12 h to 48 h. The growth was determined by measuring optical density at 660 nm with a spectrophotometer. The results were compiled to get minimum inhibitory concentrations (MIC) against each pathogen as shown in Table 2. During incubation, bacteria cells were taken at specific time intervals and the attack mechanism of the Sample on the bacteria was observed using Phase Contract Microscopy and Scanning Electron Microscopy (SEM). FIGS. 2, 3 and 4, show some of the results.

TABLE 2
MICs of the sample extract of Z. rouxii Cy7

| | MIC, mg/ml | | |
|---|---|---|---|
| | pH, | | |
| Target bacteria | unadjusted | pH, 5.5 | pH, 6.0 |
| Gram-positive bacteria | | | |
| Bacillus subtilis (IFO 13719) | 1 to 2 | 4 to 5 | >5 |
| Bacillus toyoi | 1 | 2 to 3 | >5 |
| Enterococcus faecalis (IFO 12508) | 2 | >4 | NT |
| Micrococcus luteus (IFO 12708) | 1 to 2 | 2 | >4 |
| Salmonella enteritidis (IFO 3313) | 0.7 | 1 | 2 to 3 |
| Staphylococcus aureus (IFO 14462) | 1 to 2 | 2 | NT |
| Gram-negative bacteria | | | |
| Escherichia coli (IFO 3301) | 2 | >4 | NT |
| Pseudomonas aeruginosa (IFO 12689) | 1 | 1 | NT |

### EXAMPLE 6

The experiment described in Example 5 was repeated with Micrococcus luteus (IFO 12708). The concentrations of the said Sample were 0.5 and 1.0 mg/ml respectively. The medium containing 20 μL/ml of ethyl alcohol was also used for comparison. Control I and Control II were the media with no additive. FIG. 5 shows a graph of the growth of micro-coccus luteus in accordance with Example 6. The following paragraph is an explantion of the graph shown in FIG. 5.

The bacteria grew well in the medium containing ethyl alcohol, like with the Controls; the growth reached its maximum at 48 hours. With medium containing 0..5 mg/ml of the sample, the growth was depressed up to 24 hours, after which slow growth was observed. On the other hand, with the medium containing 1.0 mg/ml, no growth was detected even though the incubation was carried out for 168 hours. After 12 hours of incubation, the Sample was added to Control I and Control II so that the concentration was 0.7 mg/ml and 1.5 mg/ml, respectively. As there was appreciable population of bacteria cells after 12 hours incubation, low concentration of 0.7 mg/ml in Control I suppressed the growth only for a few hours, after which the bacteria grew normally. On the other hand, enough concentration of 1.5 mg/ml in Control II inhibited the further growth of bacteria up to 168 hours. This test tube and the other containing 1.0 mg/ml of Sample since the beginning of the experiment other were taken out from the incubator after 168 hours incubation and the cells were harvested by centrifuging the cultural suspension. The cells were washed twice with sterilized warm water and put back into the ordinary medium with no additive. The normal growth was restored again.

The following results can be deduced from this experiment:

1) The effect of the Sample obtained by this invention is proportional to the population of cells of the bacteria: high population would be affected by using high dosage.

2) The Sample obtained from this invention can prolong its inhibition, depending on the concentration.

3) The effect of the Sample obtained from this invention is reversible; the cells attain the normal growth when put back to the ordinary medium.

### EXAMPLE 7

The same experiment described in Example 5 was repeated with bacteria Pseudomonas aeruginosa (IFO 12689). Sample concentration was 2 mg/ml and initial pH of the medium was adjusted at 4. Bacterial cells were incubated at 30° C. for up to three weeks. As pH of the medium was low enough, there was no growth. During incubation, the bacteria cells were taken occasionally with a platinum loop and their morphology was checked under light microscope. It was clear that although the cells were alive, the motility was suppressed significantly. Unlike the normally grown cells that swam in every direction, the Sample-treated cells just tilted or vibrated slowly. At the end of three-week incubation, the cells were washed twice with warm water and put back to the medium without added Sample. Growth was delayed; only after 12 hours incubation did the cells grow normally. But they were mutated; the size of the mutant cells elongated up to several times. Mutant cells formed aggregates that could be seen at the bottom of the test tube by naked eyes. The swollen mutant cells lost their mobility; they only moved slowly. There were also long chains composed of many uncleaved cells. On slant agar, the color and general appearance of the mutant cells were totally different from the normal cells. The isolated mutant cells did not change their unique characteristics even though they were transferred several times during a time span of more than eight months. This suggests that the bacterium was genetically mutated by treatment with the Sample. FIG. 6 shows the wild type cells (untreated) and mutant cells (treated) and mutant cells of P.Aeruginosa. After a one-month incubation in the Sample-added medium, the bacterial cells all died out. They did not grow when put back to ordinary medium, which suggests that the Sample has bactericidal effect with prolonged contact.

### EXAMPLE 8

The same experiment described in Example 7 was repeated with Micrococcus luteus (IFO 12708). After being incubated for 3 weeks in the medium at pH 4 with Sample concentration of 2 mg/ml, the cells were washed with warm water and put back to the ordinary medium with no added Sample. Growth was delayed, slow and poor. The cells were streaked on agar plates. Different types of mutants that had changed their color or luster were observed. One mutant lost the original bright yellow color of the wild type; it was pale yellow with no luster. Another type, shown in FIG. 7 maintained the luster but its color had changed into reddish yellow. Unlike wild type that grows in singles, pairs, quadruplets, or short chain consisting of not more than 4 or 5 cells, the mutants grow in abnormally huge aggregates, in which the giant cells did not cleave each other.

### EXAMPLE 9

The said Sample was washed with ethyl acetate to get organic acids crystallized out. The crystals mainly consisted of succinic acid, which was identified by RI and H-NMR. The remaining magma, called bio-polyphenols (BPP) was tested for its anti-oxidant activity. The BPP was dissolved in ethyl alcohol to get concentrations of 100 mg/ml to 400 mg/ml. Standard compounds of BHA, BHT and vitamin E, which are recognized as strong anti-oxidants, and ZR drink described in Example 4 were also employed for comparison. Concentration of standard compounds in ethyl alcohol was 100 mg/ml each. The assay method to determine the scavenging activity of free radical DPPH was as described below.

| | Blank | Control | |
|---|---|---|---|
| Test | (ml) | (ml) | (ml) |
| BPP/ZR/Standards | 1 | 1 | 0 |
| 0.1 M acetate buffer, pH 5.5 | 1 | 1 | 1 |
| Ethyl alcohol | 2 | 3 | 3 |
| 0.5 mM DPPH solution in ethanol | 1 | 0 | 1 |

Optical density OD was measured at 517 nm at 0 mm and 30 mm after mixing. Percent free radical scavenging activity was calculated as follows.

Percent activity=(Ac 1−(At2−Ab 1)}/Ac2×100,

Where Ac1=of control at 0 mm

At2=OD of test at 30 mm

Ab1=OD of blank at 0 mm

Ac2=OD of control at 30 mm

The results are tabulated below:

| Test item | Percent activity |
|---|---|
| Sample, 100 mg/ml | 27.2 |
| Sample, 200 mg/ml | 52.7 |
| Sample, 300 mg/ml | 73.4 |
| Sample, 400 mg/ml | 88.4 |
| ZR | 61.8 |
| BHA | 83.0 |
| BHT | 83.4 |
| Vitamin E | 83.0 |

The above results prove that the product ZR drink and the substances extracted from ZR have high activity of scavenging free radicals and they can be used as natural anti-oxidants for foods and cosmetics.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. The fermented composition of Zygosaccharomyces rouxii for use as an antimicrobial agent or as an antioxidant with varying compositions and properties according to different methods of fermentation having:
(a) pH ranging from 2 to 4
(b) Alcohol content ranging from 1 to 7% v/v
(c) Total acid content ranging from 20 to 60 milligram equivalent per liter
(d) Sugar content ranging from 2 to 40% w/v
(e) Consistency ranging from crystal clear thin liquid to highly viscous turbid slurry
(f) With or without intact yeast cells
(g) As draft, pasteurized, or sterilized

2. The fermented product of claim 1 wherein said product is straight, concentrated, tablet, or capsulated in form.

3. A method using:
(a) Single or mixed strains of Z.rouxii, either as cultured inoculum or old stock starter
(b) Any kind of medium used for microorganisms
(c) Sugars such as glucose, sucrose or maltose
(d) Sugar concentration in the medium ranging from 2 to 60%
(e) With or without employing vegetables, fruit, or herbs in the medium
(f) With or without added organic and inorganic supplements in the medium
(g) With or without aeration
(h) Fermenting temperature ranging from 25 to 35° C.
(i) Initial pH of the medium ranging from 4 to 7

4. The method of extraction and fractionation of bioactive active substances from the said fermented product.

5. The methods of application of the said fermented product of claim 1 for use as providing a food supplement, cosmetics, and drug and medicine taken orally, dermally or anally.
